(19) United States
(12) Patent Application Publication
(10) Pub. No.: US 2010/0292301 A1
(43) Pub. Date: Nov. 18, 2010

(54) NOVEL SIRNA STRUCTURES

(76) Inventors: Elena Feinstein, Rehovot (IL); Huseyin Aygun, Frankfurt am Main (DE)

Correspondence Address: FOLEY & LARDNER LLP, P.O. BOX 80278, SAN DIEGO, CA, 92138-0278, US

(21) Appl. No.: 12/528,907

(22) PCT Filed: Feb. 28, 2008
(86) PCT No.: PCT/IL08/00248

§ 371 Date: Jun. 16, 2010

Related U.S. Application Data
(60) Provisional application No. 60/904,305, filed on Feb. 28, 2007.

(51) Int. Cl. A61K 31/7088; C07H 21/04; C07K 9/00

(52) U.S. Cl. 514/44 A; 536/24.5; 530/322

(57) ABSTRACT

The present invention provides novel compounds, compositions, methods and uses for treating microvascular disorders, eye diseases and respiratory conditions based upon inhibition of a target gene. More specifically, the present invention relates to positional motifs of modified ribonucleotides useful in the design of siRNA compounds. In particular, the ribonucleotides include modified internucleotide linkages and/or modified sugar moieties. These novel siRNA compounds may be used therapeutically to treat a variety of diseases and indications.

This application claims the benefit of U.S. Provisional patent application 60/904,305 filed 28 Feb. 2007, which is hereby incorporated by reference in its entirety.

### FIELD OF THE INVENTION

The present invention relates to positional motifs of modified ribonucleotides useful in the design of siRNA compounds. In particular, the ribonucleotides include modified internucleotide linkages and/or modified sugar moieties. These novel siRNA compounds may be used therapeutically to treat a variety of diseases and indications.

### BACKGROUND OF THE INVENTION

### siRNAs and RNA Interference

RNA interference (RNAi) is a phenomenon involving double-stranded (ds) RNA-dependent gene-specific posttranscriptional silencing. Initial attempts to study this phenomenon and to manipulate mammalian cells experimentally were frustrated by an active, non-specific antiviral defense mechanism which was activated in response to long dsRNA molecules (Gil et al., Apoptosis, 2000. 5:107-114). Later, it was discovered that synthetic duplexes of 21 nucleotide RNAs could mediate gene specific RNAi in mammalian cells, without stimulating the generic antiviral defense mechanisms Elbashir et al. Nature 2001, 411:494-498 and Caplen et al. PNAS 2001, 98:9742-9747). As a result, small interfering RNAs (siRNAs), which are short double-stranded RNAs, have been widely used to inhibit gene expression and understand gene function.

RNA interference (RNAi) is mediated by small interfering RNAs (siRNAs) (Fire et al, Nature 1998, 391:806) or microRNAs (miRNAs) (Ambros V. Nature 2004, 431:350-355); and Bartel D P. Cell. 2004 116(2):281-97). The corresponding process is commonly referred to as specific post-transcriptional gene silencing when observed in plants and as quelling when observed in fungi.

An siRNA is a double-stranded RNA which down-regulates or silences (i.e. fully or partially inhibits) the expression of an endogenous or exogenous gene/mRNA. RNA interference is based on the ability of certain dsRNA species to enter a specific protein complex, where they are then targeted to complementary cellular RNAs and specifically degrades them. Thus, the RNA interference response features an endonuclease complex containing an siRNA, commonly referred to as an RNA-induced silencing complex (RISC), which mediates cleavage of single-stranded RNA having a sequence complementary to the antisense strand of the siRNA duplex. Cleavage of the target RNA may take place in the middle of the region complementary to the antisense strand of the siRNA duplex (Elbashir, et al., Genes Dev., 2001, 15:188). In more detail, longer dsRNAs are digested into short (17-29 bp) dsRNA fragments (also referred to as short inhibitory RNAs or “siRNAs”) by type III RNAses (DICER, DROSHA, etc., (see Bernstein et al., Nature, 2001, 409:363-6 and Lee et al., Nature, 2003, 425:415-9). The RISC protein complex recognizes these fragments and complementary mRNA. The whole process is culminated by endonuclease cleavage of target mRNA (McManus and Sharp, Nature Rev Genet, 2002, 3:737-47; Paddison and Hannon, Curr Opin Mol Ther. 2003, 5(3): 217-24). (For additional information on these terms and proposed mechanisms, see for example, Bernstein, et al., RNA. 2001, 7(11):1509-21; Nishikura, Cell. 2001, 107(4):415-8 and PCT Publication No. WO 01/36646).

Studies have revealed that siRNA can be effective in vivo in mammals, including humans. Specifically, Bitko et al., showed that specific siRNAs directed against the respiratory syncytial virus (RSV) nucleocapsid N gene are effective in treating mice when administered intranasally (Bitko et al., Nat. Med. 2005, 11(1):50-55). For reviews of therapeutic applications of siRNAs see for example, Barik (Mol. Med. 2005, 83: 764-773); Chakraborty (Current Drug Targets 2007 8(3):469-82) and Dykxhoorn, et al (Gene Therapy 2006, 13, 541-552). In addition, clinical studies with short siRNAs that target the VEGFR1 receptor in order to treat age-related macular degeneration (AMD) have been conducted in human patients. In studies such siRNA administered by intravitreal (intraocular) injection was found effective and safe in 14 patients tested (Kaiser, Am J. Opthalmol. 2006 142(4):660-8).

There remains an unmet need for therapeutic double stranded oligomeric compounds exhibiting good in vivo stability and activity.

Due to the difficulty in identifying and obtaining regulatory approval for chemical drugs for the treatment of diseases, the molecules of the present invention offer an advantage in that they are non-toxic and may be formulated as pharmaceutical compositions for treatment of any disease.

### SUMMARY OF THE INVENTION

The present invention relates to novel structural motifs applicable to double stranded oligonucleotides useful to inhibit any gene. The structural motifs are based on modification of ribonucleotides at certain positions in either or both of the sense and antisense strands. Without wishing to be bound to theory, the modified oligoribonucleotide down-regulates the expression of a target gene by the mechanism of RNA interference. The invention also provides a pharmaceutical composition comprising such oligoribonucleotides and methods of treating disease comprising administering the oligoribonucleotide to a subject in need thereof.

Accordingly, in one aspect the present invention provides a compound having a structure set forth below:

| | |
|---|---|
| 5′ (N)ₓ-Z 3′ | (antisense strand) |
| 3′ Z′-(N′)_{y} 5′ | (sense strand) |

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein each of x and y is an integer between 18 and 40; wherein each of Z and Z′ may be present or absent, but if present is 1-5 consecutive nucleotides covalently attached at the 3′ terminus of the strand in which it is present;
wherein the sequence of (N)x is complementary to the sequence of (N′)y;
wherein the sequence of (N′)y is present within the mRNA of a target gene;
wherein at least one of (N)x or (N′)y comprises a modification selected from the group consisting of internucleotide modification and an L-nucleotide;
and wherein the internucleotide modification is a 5′-2′ internucleotide bridge or an alpha phosphate modification selected from the group consisting of thiophosphate and triester.

In some embodiments the internucleotide modification is present in any one of the motifs selected from the group consisting of
a) an internucleotide modification between nucleotides at positions 1-19 of (N′)y;
b) an internucleotide modification between nucleotides at positions 1-19 of (N)x;
c) an internucleotide modification between nucleotides at positions 9-11 of the sense strand and positions 1-9 and 11-19 of the antisense strand;
d) an internucleotide modification between nucleotides at positions 1-2, 3-4, 5-6, 7-8, 12-13, 14-15, 16-17 and 18-19 of the sense strand and positions 2-3, 4-5, 6-7, 8-9 11-12, 13-14, 15-16 and 17-18 of the antisense strand;
e) an internucleotide modification between nucleotides at positions 1-2, 3-4, 5-6, 7-8, 14-15, 16-17 and 18-19 of the sense strand and positions 2-3, 4-5, 6-7, 13-14, 15-16 and 17-18 of the antisense strand;
f) an internucleotide modification between nucleotides at positions 1-2, 3-4, 16-17 and 18-19 of the sense strand and 2-3, 4-5, 15-16 and 17-18 of the antisense strand;
g) an internucleotide modification between nucleotides at positions 1-2, 3-4, 16-17 and 18-19 of the sense strand and 2-3 and 17-18 of the antisense strand;
h) an internucleotide modification between nucleotides at positions 2-3 and 17-18 of the sense strand and 1-2, 3-4, 16-17 and 18-19 of the antisense strand;
i) an internucleotide modification between nucleotides at positions 1-2, 6-7, 13-14 and 18-19 of the sense strand and 5-6 and 14-15 of the antisense strand;
j) an internucleotide modification between nucleotides at positions 6-7 and 13-14 of the sense strand and 1-2, 5-6, 14-15 and 18-19 of the antisense strand;
k) an internucleotide modification between nucleotides at positions 1-2, 4-5, 6-7, 13-14, 15-16 and 18-19 of the sense strand and 5-6, 7-8, 12-13 and 14-15 of the antisense strand; and
l) an internucleotide modification between nucleotides at positions 4-5, 6-7, 13-14 and 15-16 of the sense strand and 1-2, 5-6, 7-8, 12-13, 14-15 and 18-19 of the antisense strand.

In certain embodiments the internucleotide modification is a 5′-2′ bridge. In other embodiments the alpha phosphate modification is selected from the group consisting of phospho-ethyl triester, phospho-propyl triester and phospho-butyl triester.

In another embodiment one of (N)x and (N′)y comprises unmodified ribonucleotides and L-nucleotides wherein the motif is selected from the group consisting of
a) An L-nucleotide at each of positions 1-19 of the sense strand (i.e., a sense strand composed only of mirror nucleotides);
b) An L-nucleotide at each of positions 1-19 of the antisense strand (i.e., an antisense strand composed only of mirror nucleotides);
c) An L-nucleotide at each of positions 9-11 of the sense strand and at each of positions 1-9 and 11-19 of the antisense strand;
d) An L-nucleotide at each of positions 1-8, and 12-19 of the sense strand and at each of positions 2-9 and 11-18 of the antisense strand;
e) An L-nucleotide at each of positions 1-8 and 14-19 of the sense strand and at each of positions 2-7 and 13-18 of the antisense strand;
f) An L-nucleotide at each of positions 1-4 and 16-19 of the sense strand and at each of positions 2-5 and 15-18 of the antisense strand;
g) An L-nucleotide at each of positions 1-4, 16-19 of the sense strand and at each of positions 2-3 and 17-18 of the antisense strand;
h) An L-nucleotide at each of positions 2-3 and 17-18 of the sense strand and at each of positions 1-4 and 16-19 of the antisense strand;
i) An L-nucleotide at each of positions 1-2, 6-7, 13-14 and 18-19 of the sense strand and at each of positions 5-6 and 14-15 of the antisense strand;
j) An L-nucleotide at each of positions 6-7 and 13-14 of the sense strand and at each of positions 1-2, 5-6, 14-15 and 18-19 of the antisense strand;
k) An L-nucleotide at each of positions 1-2, 4-7, 13-16 and 18-19 of the sense strand and at each of positions 5-8, 12-15 of the antisense strand;
l) An L-nucleotide at each of positions 4-7 and 13-16 of the sense strand and at each of positions 1-2, 5-8, 12-15 and 18-19 of the antisense strand.

In various embodiments x=y. In certain preferred embodiments x=y=19 or x=y=23. In some embodiments Z═Z′=0.

In a second aspect the present invention provides a pharmaceutical composition comprising the oligonucleotide compound of the invention; and a pharmaceutically acceptable carrier.

In another aspect the present invention provides a method of treating a patient suffering from a disease or adverse condition, comprising administering to the patient the oligoribonucleotide typically as a pharmaceutical composition, in a therapeutically effective amount so as to thereby treat the patient. In certain preferred embodiments the oligonucleotide compound of the invention is administered as a naked compound as defined below.

The present invention also relates to functional nucleic acids comprising various modifications, and their use for the manufacture of a medicament useful in for the treating various diseases and disorders.

### BRIEF DESCRIPTION OF THE FIGURES

FIG. 1 demonstrates the position of backbone modification(s) on exemplary molecules of the present invention;

FIG. 2 is an example of nucleotide modification used in the molecules of the present invention;

FIG. 3 is an example of sugar modifications used in the molecules of the present invention;

FIG. 4 is an example of backbone modifications included in the molecules of the present invention.

### DETAILED DESCRIPTION OF THE INVENTION

The present invention relates to modified oligonucleotides and oligoribonucleotides which possess therapeutic properties. In particular, the present invention discloses oligoribonucleotides which encode an inhibitory RNA molecule such as an siRNA. The siRNAs of the present invention possess novel structures and novel modification combinations which have the advantage of increased stability and minimized toxicity; the novel modifications of the siRNAs of the present invention can be beneficially applied to any siRNA or other RNAi-inducing nucleic acid molecule.

### DEFINITIONS

For convenience certain terms employed in the specification, examples and claims are described herein.

It is to be noted that, as used herein, the singular forms “a”, “an” and “the” include plural forms unless the content clearly dictates otherwise.

Where aspects or embodiments of the invention are described in terms of Markush groups or other grouping of alternatives, those skilled in the art will recognize that the invention is also thereby described in terms of any individual member or subgroup of members of the group.

In this context by “backbone modification” is meant “internucleotide modification” In naturally occurring polynucleotides, the polymer of nucleotides has a backbone in which sugars and phosphate groups are joined by ester bonds. Thus, the “backbone” of a nucleic acid molecule comprises alternating phosphate and sugar residues. Thus “backbone modification” and “internucleotide modification” are used interchangeably herein. According to the many modifications as disclosed herein, molecules of the present invention are not limited to naturally occurring backbones and many comprise backbones in which the ester bond is replaced with a different type of bond, such as triester or thioate, or backbones in which the conformation of the bond is altered e.g. 5′-2′ internucleotide bridge.

“Target mRNA” encompasses vertebrate mRNA such as mammalian mRNA including human, and invertebrates, protozoa, plant, fungi, bacterial and viral RNA, inter alia.

Thus, in one embodiment, the present invention provides for an oligonucleotide comprising consecutive nucleotides which encode an inhibitory nucleic acid molecule. The oligonucleotide may contain modified nucleotides such as DNA, LNA, PNA, arabinoside or one or more mirror nucleotide. The oligonucleotide may further comprise 2′OMethyl or 2′Fluoro or 2′Oallyl or any other 2′ modification, optionally on alternate positions. Other stabilizing patterns which do not significantly reduce the enzymatic activity are also possible (i.e. terminal modifications). The backbone of the active part of tandem oligonucleotides may comprise phosphate-D-ribose entities but may also contain thiophosphate-D-ribose entities, triester, thioate, 5′-2′ bridged backbone. Terminal modifications on the 5′ and/or 3′ part of the tandem oligonucleotides are also possible. Such terminal modifications may be lipids, peptides, sugars or other molecules.

In an additional embodiment, the present invention provides for a double stranded oligomeric compound wherein each strand consists of 18-40 nucleotides and preferably 19 mer (or 20 mer or 21 mer or 23 mer) inhibitory oligonucleotide, preferably oligoribonucleotide, comprising one or more backbone modification selected from the group consisting of thiophosphate, triester and 5′-2′ bridge. The oligonucleotide may be modified according to one of the following motifs:
a) an oligonucleotide backbone modification between nucleotides at positions 1-19 of the sense strand;
b) an oligonucleotide backbone modification between nucleotides at positions 1-19 of the antisense strand;
c) an oligonucleotide backbone modification between nucleotides at positions 9-11 of the sense strand and 1-9 and 11-19 of the antisense strand;
d) an oligonucleotide backbone modification between nucleotides at positions 1-2, 3-4, 5-6, 7-8, 12-13, 14-15, 16-17 and 18-19 of the sense strand and 2-3, 4-5, 6-7, 8-9 11-12, 13-14, 15-16 and 17-18 of the antisense strand;
e) an oligonucleotide backbone modification between nucleotides at positions 1-2, 3-4, 5-6, 7-8, 14-15, 16-17 and 18-19 of the sense strand and 2-3, 4-5, 6-7, 13-14, 15-16 and 17-18 of the antisense strand;
f) an oligonucleotide backbone modification between nucleotides at positions 1-2, 3-4, 16-17 and 18-19 of the sense strand and 2-3, 4-5, 15-16 and 17-18 of the antisense strand;
g) an oligonucleotide backbone modification between nucleotides at positions 1-2, 3-4, 16-17 and 18-19 of the sense strand and 2-3 and 17-18 of the antisense strand;
h) an oligonucleotide backbone modification between nucleotides at positions 2-3 and 17-18 of the sense strand and 1-2, 3-4, 16-17 and 18-19 of the antisense strand;
i) an oligonucleotide backbone modification between nucleotides at positions 1-2, 6-7, 13-14 and 18-19 of the sense strand and 5-6 and 14-15 of the antisense strand;
j) an oligonucleotide backbone modification between nucleotides at positions 6-7 and 13-14 of the sense strand and 1-2, 5-6, 14-15 and 18-19 of the antisense strand;
k) an oligonucleotide backbone modification between nucleotides at positions 1-2, 4-5, 6-7, 13-14, 15-16 and 18-19 of the sense strand and 5-6, 7-8, 12-13 and 14-15 of the antisense strand; or
l) an oligonucleotide backbone modification between nucleotides at positions 4-5, 6-7, 13-14 and 15-16 of the sense strand and 1-2, 5-6, 7-8, 12-13, 14-15 and 18-19 of the antisense strand.

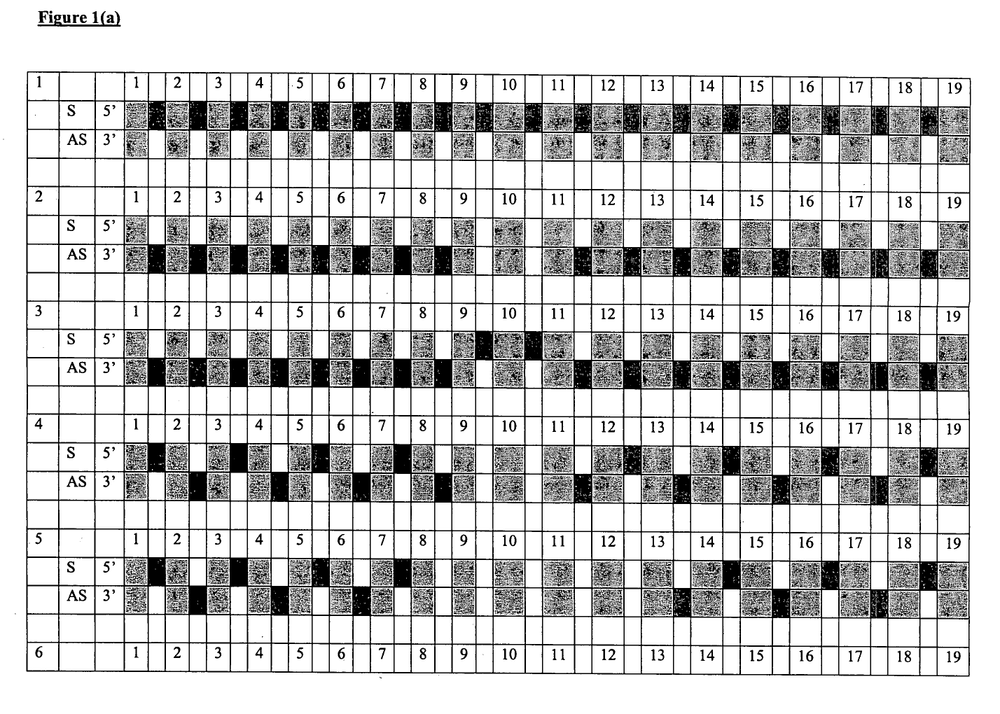

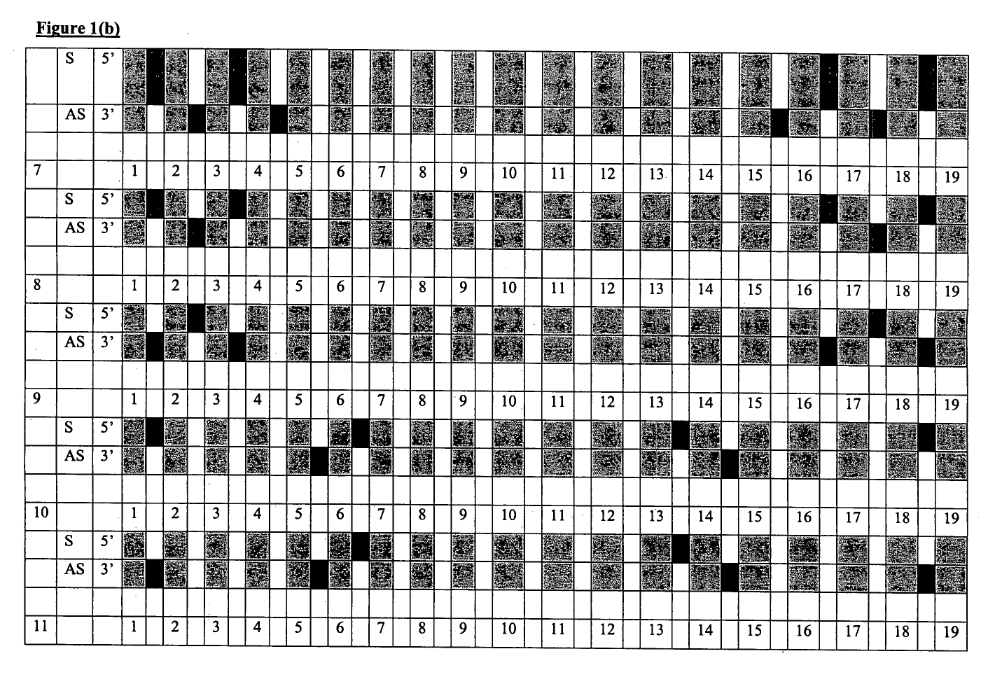

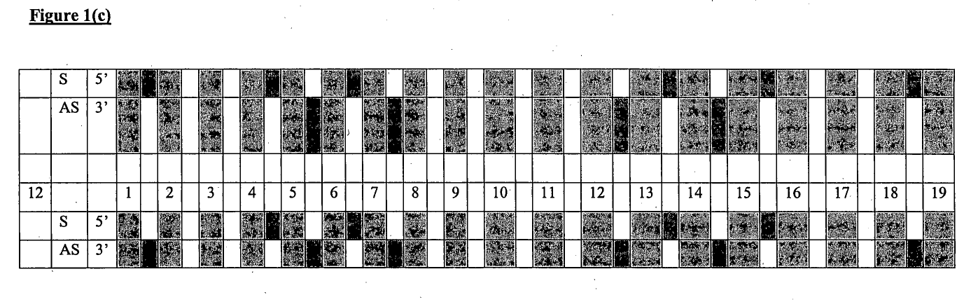

The modification patterns (a through l) are presented graphically in FIG. 1, wherein the darker squares represent modified internucleotide linkages and the white squares represent non-modified internucleotide linkages.

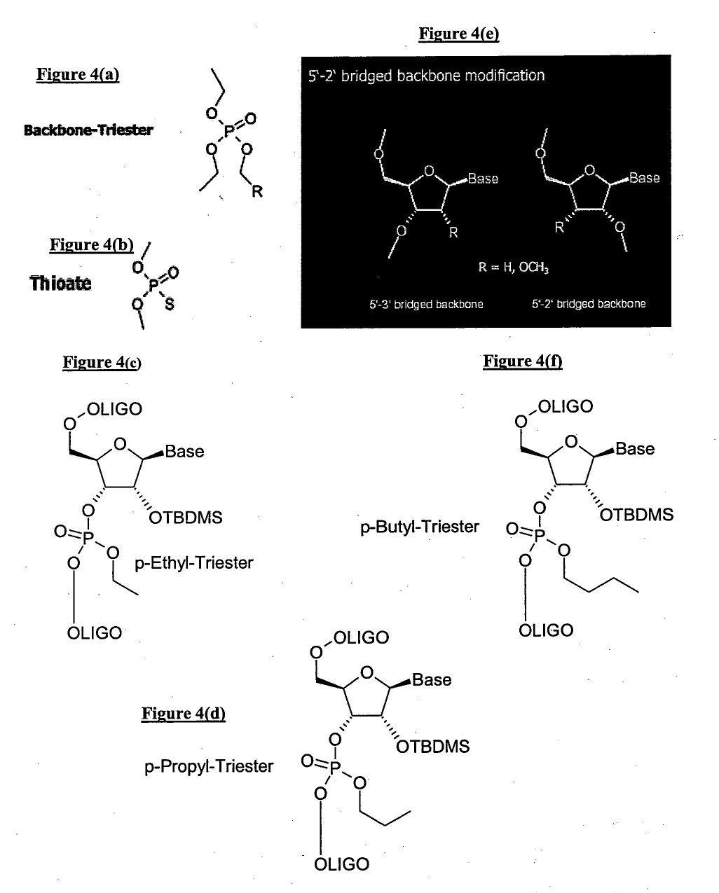

The modification of choice is any modification as disclosed herein, including Ethyl (resulting in a phospho-ethyl triester); Propyl (resulting in a phospho-propyl triester); and Butyl (resulting in a phospho-butyl triester). Other possible backbone modifications include thioate modifications or 5′-2′ bridged backbone modifications (see FIG. 4).

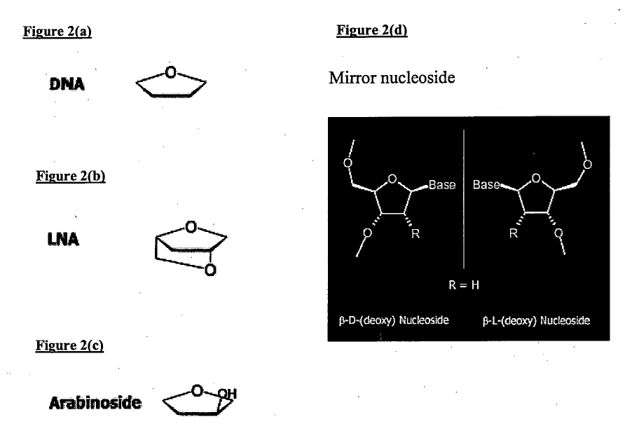

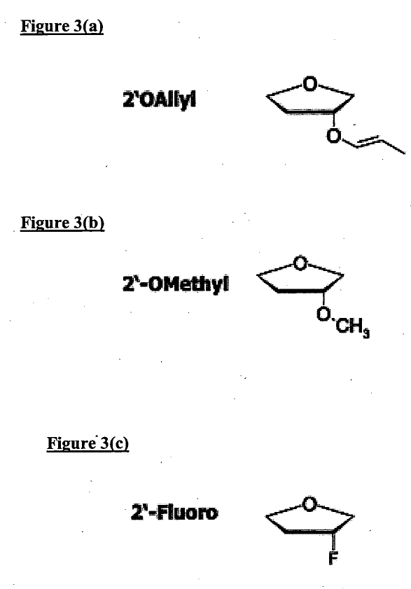

Additional modifications which can be present in the above molecules possessing any one of the patterns of backbone modifications as described in a-l above and further include nucleotide modifications, peptide nucleic acid (PNA), morpholino and locked nucleic acid (LNA), glycol nucleic acid (GNA), threose nucleic acid (TNA), arabinoside, and mirror nucleotide (for example, beta-L-deoxyribonucleotide instead of the naturally occurring beta-D-deoxyribonucleotide; see FIG. 2).

Further, said molecules may additionally contain modifications on the sugar, such as 2′ alkoxy including 2′O-methyl (2′OMe); 2′ fluoro; 2′O-allyl; 2′ amine and additional sugar modifications are discussed herein.

Thus, the novel modified inhibitory nucleic acid molecules of the present invention may posses one or more backbone modification, one or more modified nucleotide and one or more nucleotide having one or more modified position on its sugar. Any possible combination of the modifications discussed herein is possible, and constitutes a part of the present invention.

Further, inhibitory nucleic acid molecules which contain mirror nucleotides are a part of the present invention.

In the context of the present invention, the term “mirror” nucleotide is used interchangeably with “L-nucleotide” and refers to a nucleotide with reversed chirality to the naturally occurring or commonly employed nucleotide, i.e., a mirror image of the naturally occurring or commonly employed nucleotide. See for example U.S. Pat. No. 6,602,858, which discloses nucleic acid catalysts comprising at least one L-nucleotide substitution. In some embodiments the L-nucleotide is L-RNA. In certain preferred embodiments the L-nucleotide is L-DNA.

The inhibitory nucleic acid molecules of the present invention is preferably a double stranded oligomeric compound wherein each strand is 19, 20 or 21 ribonucleotides in length. The structural motifs are also useful for longer oligomers, for example, 23, 24, 25 and up to oligomers 40 nucleotides long and may comprise one or more mirror nucleotide according to the following patterns:
a) A mirror nucleotide at positions 1-19 of the sense strand (i.e., a sense strand composed only of mirror nucleotides);
b) A mirror nucleotide at positions 1-19 of the antisense strand (i.e., an antisense strand composed only of mirror nucleotides);
c) A mirror nucleotide at positions 9-11 of the sense strand and 1-9 and 11-19 of the antisense strand;
d) A mirror nucleotide at positions 1-2, 3-4, 5-6, 7-8, 12-13, 14-15, 16-17 and 18-19 of the sense strand and 2-3, 4-5, 6-7, 8-9 11-12, 13-14, 15-16 and 17-18 of the antisense strand;
e) A minor nucleotide at positions 1-2, 3-4, 5-6, 7-8, 14-15, 16-17 and 18-19 of the sense strand and 2-3, 4-5, 6-7, 13-14, 15-16 and 17-18 of the antisense strand;
f) A mirror nucleotide at positions 1-2, 3-4, 16-17 and 18-19 of the sense strand and 2-3, 4-5, 15-16 and 17-18 of the antisense strand;
g) A mirror nucleotide at positions 1-2, 3-4, 16-17 and 18-19 of the sense strand and 2-3 and 17-18 of the antisense strand;
h) A mirror nucleotide at positions 2-3 and 17-18 of the sense strand and 1-2, 3-4, 16-17 and 18-19 of the antisense strand;
i) A mirror nucleotide at positions 1-2, 6-7, 13-14 and 18-19 of the sense strand and 5-6 and 14-15 of the antisense strand;
j) A mirror nucleotide at positions 6-7 and 13-14 of the sense strand and 1-2, 5-6, 14-15 and 18-19 of the antisense strand;
k) A mirror nucleotide at positions 1-2, 4-5, 6-7, 13-14, 15-16 and 18-19 of the sense strand and 5-6, 7-8, 12-13 and 14-15 of the antisense strand;
l) A mirror nucleotide at positions 4-5, 6-7, 13-14 and 15-16 of the sense strand and 1-2, 5-6, 7-8, 12-13, 14-15 and 18-19 of the antisense strand.

Any other pattern of mirror-nucleotide containing inhibitory nucleic acid molecule is also possible.

The above mirror nucleoside containing inhibitory nucleic acid molecules may also comprise one or more additional modified nucleoside, such as DNA, LNA, PNA, arabinoside or any other possible modified nucleotide described herein. They may also comprise one or more sugar modifications, such as 2′ alkoxy preferably a 2′O methyl, 2′ fluoro, 2′O-allyl, 2′ amine, or any other possible sugar modification disclosed herein.

Additionally, any of the molecules of the present invention may also comprise one or more terminal modification, optionally selected from a nucleotide, a di-nucleotide, an oligonucleotide, a lipid, a peptide, a sugar or an amine.

Further, the inhibitory nucleic acid molecules of the present invention may comprise one or more gaps and/or one or more nicks and/or one or more mismatches. Without being bound by theory, gaps, nicks and mismatches may have the advantage of partially destabilizing the nucleic acid/siRNA, so that it may be more easily processed by endogenous cellular machinery such as DICER, DROSHA or RISC into its inhibitory components.

In the context of the present invention, a gap in a nucleic acid means that the molecule is missing one or more nucleotide at the site of the gap, while a nick in a nucleic acid means that there are no missing nucleotides, but rather, there is no phospho-diester or other bond between 2 adjacent nucleotides at the site of the nick. Any of the molecules of the present invention may contain one or more gaps and/or one or more nicks.

Further provided by the present invention is an siRNA encoded by any of the molecules disclosed herein and a pharmaceutical composition comprising any of the molecules disclosed herein and a pharmaceutically acceptable carrier.

Said pharmaceutical compositions may be used in the treatment of a variety of diseases and indications and, as discussed herein, they have a particular advantage in that they increase efficacy and minimize side effects. In particular, the pharmaceutical compositions of the present invention can be used to treat a respiratory disorder such as COPD, a microvascular disorder such as acute renal failure or diabetic retinopathy and in particular an eye disease such as ocular scarring or macular degeneration. In a particular embodiment the siRNA targets gene 801 as described in co-assigned PCT patent publication No. WO06/023544A2, which is hereby incorporated by reference in its entirety.

“Respiratory disorder” refers to conditions, diseases or syndromes of the respiratory system including but not limited to pulmonary disorders of all types including chronic obstructive pulmonary disease (COPD), emphysema, chronic bronchitis, asthma and lung cancer, inter alia. Emphysema and chronic bronchitis may occur as part of COPD or independently. In a particular embodiment the siRNA targets gene 801 as described in co-assigned PCT patent publication No. WO06/023544A2

“Microvascular disorder” refers to any condition that affects microscopic capillaries and lymphatics, in particular vasospastic diseases, vasculitic diseases and lymphatic occlusive diseases. Examples of microvascular disorders include, inter alia: eye disorders such as Amaurosis Fugax (embolic or secondary to SLE), apla syndrome, Prot CS and ATIII deficiency, microvascular pathologies caused by IV drug use, dysproteinemia, temporal arteritis, anterior ischemic optic neuropathy, optic neuritis (primary or secondary to autoimmune diseases), glaucoma, von hippel lindau syndrome, corneal disease, corneal transplant rejection cataracts, Eales' disease, frosted branch angiitis, encircling buckling operation, uveitis including pars planitis, choroidal melanoma, choroidal hemangioma, optic nerve aplasia; retinal conditions such as retinal artery occlusion, retinal vein occlusion, retinopathy of prematurity, HIV retinopathy, Purtscher retinopathy, retinopathy of systemic vasculitis and autoimmune diseases, diabetic retinopathy, hypertensive retinopathy, radiation retinopathy, branch retinal artery or vein occlusion, idiopathic retinal vasculitis, aneurysms, neuroretinitis, retinal embolization, acute retinal necrosis, Birdshot retinochoroidopathy, long-standing retinal detachment; systemic conditions such as Diabetes mellitus, diabetic retinopathy (DR), diabetes-related microvascular pathologies (as detailed herein), hyperviscosity syndromes, aortic arch syndromes and ocular ischemic syndromes, carotid-cavernous fistula, multiple sclerosis, systemic lupus erythematosus, arteriolitis with SS-A autoantibody, acute multifocal hemorrhagic vasculitis, vasculitis resulting from infection, vasculitis resulting from Behcet's disease, sarcoidosis, coagulopathies, neuropathies, nephropathies, microvascular diseases of the kidney, and ischemic microvascular conditions, inter alia. Microvascular disorders may comprise a neovascular element. The term “neovascular disorder” refers to those conditions where the formation of blood vessels (neovascularization) is harmful to the patient. Examples of ocular neovascularization include: retinal diseases (diabetic retinopathy, diabetic Macular Edema, chronic glaucoma, retinal detachment, and sickle cell retinopathy); rubeosis iritis; proliferative vitreo-retinopathy; inflammatory diseases; chronic uveitis; neoplasms (retinoblastoma, pseudoglioma and melanoma); Fuchs' heterochromic iridocyclitis; neovascular glaucoma; corneal neovascularization (inflammatory, transplantation and developmental hypoplasia of the iris); neovascularization following a combined vitrectomy and lensectomy; vascular diseases (retinal ischemia, choroidal vascular insufficiency, choroidal thrombosis and carotid artery ischemia); neovascularization of the optic nerve; and neovascularization due to penetration of the eye or contusive ocular injury. All these neovascular conditions may be treated using the compounds and pharmaceutical compositions of the present invention.

“Eye disease” refers to refers to conditions, diseases or syndromes of the eye including but not limited to any conditions involving choroidal neovascularization (CNV), wet and dry AMD, ocular histoplasmosis syndrome, angiod streaks, ruptures in Bruch's membrane, myopic degeneration, ocular tumors, ocular scarring, retinal degenerative diseases and retinal vein occlusion (RVO).

The pharmaceutical composition is in its various embodiments is adapted for administration in various ways. Such administration comprises systemic and local administration as well as oral, subcutaneous, parenteral, intravenous, intraarterial, intramuscular, intraperitonial, intranasal, and intrategral.

It will be acknowledged by those skilled in the art that the amount of the pharmaceutical composition and the respective nucleic acid, respectively, depends on the clinical condition of the individual patient, the site and method of administration, scheduling of administration, patient age, sex, bodyweight and other factors known to medical practitioners. The pharmaceutically effective amount for purposes of prevention and/or treatment is thus determined by such considerations as are known in the medical arts. Preferably, the amount is effective to achieve improvement including but limited to improve the diseased condition or to provide for a more rapid recovery, improvement or elimination of symptoms and other indicators as are selected as appropriate measures by those skilled in the medical arts.

In a preferred embodiment, the pharmaceutical composition according to the present invention may comprise other pharmaceutically active compounds. Preferably, such other pharmaceutically active compounds are selected from the group comprising compounds which allow for uptake intracellular cell delivery, compounds which allow for endosomal release, compounds which allow for, longer circulation time and compounds which allow for targeting of endothelial cells or pathogenic cells. Preferred compounds for endosomal release are chloroquine, and inhibitors of ATP dependent H⁺ pumps. The pharmaceutical composition is preferably formulated so as to provide for a single dosage administration or a multi-dosage administration. The compound may also be administered as a naked oligonucleotide. The term “naked” DNA or RNA refers to sequences that are free from any delivery vehicle that acts to assist, promote or facilitate entry into the cell, including viral sequences, viral particles, liposome formulations, lipofectin or precipitating agents and the like. However, the polynucleotides of the invention can also be delivered in liposome formulations and lipofectin formulations and the like can be prepared by methods well known to those skilled in the art. Such methods are described, for example, in U.S. Pat. Nos. 5,593,972, 5,589,466, and 5,580,859, which are herein incorporated by reference.

For further information on dosage, formulation and delivery of the compounds of the present invention see Example 3.

“Treating a disease” refers to administering a therapeutic substance effective to ameliorate symptoms associated with a disease, to lessen the severity or cure the disease, or to prevent the disease from occurring. The term “disease” comprises any illness or adverse condition.

A “therapeutically effective dose” refers to an amount of a pharmaceutical compound or composition which is effective to achieve an improvement in a patient or his physiological systems including, but not limited to, improved survival rate, more rapid recovery, or improvement or elimination of symptoms, and other indicators as are selected as appropriate determining measures by those skilled in the art.

An “inhibitor” is a compound which is capable of inhibiting the activity of a gene or the product of such gene to an extent sufficient to achieve a desired biological or physiological effect. Such inhibitors include substances that affect the transcription or translation of the gene as well as substances that affect the activity of the gene product. Examples of such inhibitors may include, inter alia: polynucleotides such as antisense (AS) fragments and siRNA, polypeptides such as dominant negatives, antibodies, and enzymes; catalytic RNAs such as ribozymes; and chemical molecules with a low molecular weight e.g. a molecular weight below 2000 daltons. By “small interfering RNA” (siRNA) is meant an RNA molecule which decreases or silences (prevents) the expression of a gene/mRNA of its endogenous cellular counterpart. The term is understood to encompass “RNA interference” (RNAi). RNA interference (RNAi) refers to the process of sequence-specific post transcriptional gene silencing in mammals mediated by small interfering RNAs (siRNAs) (Fire et al, 1998, Nature 391, 806). The corresponding process in plants is commonly referred to as specific post transcriptional gene silencing or RNA silencing and is also referred to as quelling in fungi. The RNA interference response may feature an endonuclease complex containing an siRNA, commonly referred to as an RNA-induced silencing complex (RISC), which mediates cleavage of single-stranded RNA having sequence complementary to the antisense strand of the siRNA duplex. Cleavage of the target RNA may take place in the middle of the region complementary to the antisense strand of the siRNA duplex (Elbashir et al 2001, Genes Dev., 15, 188). For recent information on these terms and proposed mechanisms, see Bernstein E., Denli A M., Hannon G J: The rest is silence. RNA. 2001 November; 7(11):1509-21; and Nishikura K.: A short primer on RNAi: RNA-directed RNA polymerase acts as a key catalyst. Cell. 2001 Nov. 16; 107(4):415-8.

During recent years, RNAi has emerged as one of the most efficient methods for inactivation of genes (Nature Reviews, 2002, v.3, p. 737-47; Nature, 2002, v.418, p. 244-51). As a method, it is based on the ability of dsRNA species to enter a specific protein complex, where it is then targeted to the complementary cellular RNA and specifically degrades it. In more detail, dsRNAs are digested into short (17-29 bp) inhibitory RNAs (siRNAs) by type III RNAses (DICER, Drosha, etc) (Nature, 2001, v.409, p. 363-6; Nature, 2003, 425, p. 415-9). These fragments and complementary mRNA are recognized by the specific RISC protein complex. The whole process is culminated by endonuclease cleavage of target mRNA (Nature Reviews, 2002, v.3, p. 737-47; Curr Opin Mol. Ther. 2003 June; 5(3):217-24).

For disclosure on how to design and prepare siRNA to known genes see for example Chalk A M, Wahlestedt C, Sonnhammer E L. Improved and automated prediction of effective siRNA Biochem. Biophys. Res. Commun. 2004 Jun. 18; 319(1):264-74; Sioud M, Leirdal M., Potential design rules and enzymatic synthesis of siRNAs, Methods Mol Biol. 2004; 252:457-69; Levenkova N, Gu Q, Rux J J.: Gene specific siRNA selector Bioinformatics. 2004 Feb. 12; 20(3):430-2. and Ui-Tei K, Naito Y, Takahashi F, Haraguchi T, Ohki-Hamazaki H, Juni A, Ueda R, Saigo K., Guidelines for the selection of highly effective siRNA sequences for mammalian and chick RNA interference Nucleic Acids Res. 2004 Feb. 9; 32(3):936-48. See also Liu Y, Braasch D A, Nulf C J, Corey D R. Efficient and isoform-selective inhibition of cellular gene expression by peptide nucleic acids Biochemistry, 2004 Feb. 24; 43(7):1921-7. See also PCT publications WO 2004/015107 (Atugen) and WO 02/44321 (Tuschl et al), and also Chiu Y L, Rana T M. siRNA function in RNAi: a chemical modification analysis, RNA 2003 September; 9(9):1034-48 and U.S. Pat. Nos. 5,898,031 and 6,107,094 (Crooke) for production of modified/more stable siRNAs.

For delivery of siRNAs, see, for example, Shen et al (FEBS letters 539: 111-114 (2003)), Xia et al., Nature Biotechnology 20:. 1006-1010 (2002), Reich et al., Molecular Vision 9: 210-216 (2003), Sorensen et al. (J. Mol. Biol. 327: 761-766 (2003), Lewis et al., Nature Genetics 32: 107-108 (2002) and Simeoni et al., Nucleic Acids Research 31, 11: 2717-2724 (2003). siRNA has recently been successfully used for inhibition in primates; for further details see Tolentino et al., Retina 24(1) February 2004 pp 132-138.

In connection with the compounds of the present invention, the modifications as discussed above may be selected from the group comprising sugar modifications such as amino, fluoro, alkoxy (including LNAs [linked nucleic acids]—which are circularized alkoxy modifications) or alkyl and base modifications such as 5-Alkyl-pyrimidines, 7-Deaza-purines, 8-Alkyl-purines or many other base modifications.

The double stranded structure of the siRNA may be blunt ended, on one or both sides. More specifically, the double stranded structure may be blunt ended on the double stranded structure's side which is defined by the 5′-end of the first strand and the 3′-end of the second strand, or the double stranded structure may be blunt ended on the double stranded structure's side which is defined by at the 3′-end of the first strand and the 5′-end of the second strand.

Additionally, at least one of the two strands may have an overhang of at least one nucleotide at the 5′-end; the overhang may consist of at least one deoxyribonucleotide. At least one of the strands may also optionally have an overhang of at least one nucleotide at the 3′-end.

The length of the double-stranded structure of the siRNA is typically from about 17 to 25 and more preferably 19-23 bases. Further, the length of said first strand and/or the length of said second strand may independently from each other be selected from the group comprising the ranges of from about 15 to about 23 bases, 17 to 21 bases, 19-21 bases and 18 or 19 bases.

Additionally, the complementarily between said first strand and the target nucleic acid may be perfect, or the duplex formed between the first strand and the target nucleic acid may comprise at least 15 nucleotides wherein there is one mismatch or two mismatches between said first strand and the target nucleic acid forming said double-stranded structure.

Further the sense strand of the siRNA may comprise eight to twelve, preferably nine to eleven, groups of modified nucleotides, and the antisense strand may comprise seven to eleven, preferably eight to ten, groups of modified nucleotides.

The sense strand and the antisense strand may be linked by a loop structure, which may be comprised of a non-nucleic acid polymer such as, inter alia, polyethylene glycol. Alternatively, the loop structure may be comprised of a nucleic acid. The loop structure may additionally be comprised of amino acids or PNAs.

Further, the 5′-terminus of the sense strand of the siRNA may be linked to the 3′-terminus of the antisense strand, or the 3′-end of the sense strand may be linked to the 5′-terminus of the antisense strand, said linkage being via a nucleic acid linker typically having a length between 3-50 residues.

In further embodiments, the siRNAs of the present invention, the various possible properties of which are described herein, are linked together by a variety of linkers as described above, such that a molecule which comprises two or more siRNA moieties is created. Such molecules are novel and may be used to treat a variety of indications, as described herein.

The invention provides a molecule comprising a compound having the structure:

| | |
|---|---|
| 5′ (N)ₓ-Z 3′ | (antisense strand) |
| 3′ Z′-(N′)_{y} 5′ | (sense strand) |

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein each of x and y is an integer between 18 and 40;
wherein each of Z and Z′ may be present or absent, but if present is 1-5 consecutive nucleotides covalently attached at the 3′ terminus of the strand in which it is present;
wherein the sequence of (N)x is complementary to the sequence of (N′)y;
wherein the sequence of (N′)y is present within the mRNA of a target gene;
wherein at least one of (N)x or (N′)y comprises a modification selected from the group consisting of internucleotide modification and an L-nucleotide;
and wherein the internucleotide modification is 5′-2′ bridge or an alpha phosphate modification selected from the group consisting of thiophosphate and triester.

In particular, the invention provides the above compound wherein the covalent bond is a phosphodiester bond, wherein x=y or y-1, preferably wherein x=y=19 or 20; or x=20 and y=19; or x=19 and y=20, wherein Z and Z′ are both absent, wherein at least one ribonucleotide is modified in its sugar residue at the 2′ position, wherein the moiety at the 2′ position is methoxy (2′-O-Methyl) wherein alternating ribonucleotides are modified in both the antisense and the sense strands and wherein the ribonucleotides at the 5′ and 3′ termini of the antisense strand are modified in their sugar residues, and the ribonucleotides at the 5′ and 3′ termini of the sense strand are unmodified in their sugar residues.

In an additional embodiment the present invention provides a compound having a structure set forth below:

| | |
|---|---|
| 5′ (N)ₓ-Z 3′ | (antisense strand) |
| 3′ Z′-(N′)_{y} 5′ | (sense strand) |

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein each of x and y is an integer between 19 and 23;
wherein each of Z and Z′ may be present or absent, but if present is 1-5 consecutive nucleotides covalently attached at the 3′ terminus of the strand in which it is present;
wherein the sequence of (N)x is complementary to the sequence of (N′)y;
wherein the sequence of (N′)y is present within the mRNA of a target gene;
wherein at least one of (N)x or (N′)y comprises a modification selected from the group consisting of internucleotide modification and an L-nucleotide;
and wherein the internucleotide modification is 5′-2′ bridge or an alpha phosphate modification selected from the group consisting of thiophosphate and triester.

The alpha phosphate modification may be present in any one of the motifs selected from the group consisting of
a) an internucleotide modification between nucleotides at positions 1-19 of (N′)y;
b) an internucleotide modification between nucleotides at positions 1-19 of (N)x;
c) an internucleotide modification between nucleotides at positions 9-11 of (N′)y and positions 1-9 and 11-19 of (N)x;
d) an internucleotide modification between nucleotides at positions 1-2, 3-4, 5-6, 7-8, 12-13, 14-15, 16-17 and 18-19 of (N′)y and positions 2-3, 4-5, 6-7, 8-9 11-12, 13-14, 15-16 and 17-18 of (N)x;
e) an internucleotide modification between nucleotides at positions 1-2, 3-4, 5-6, 7-8, 14-15, 16-17 and 18-19 of (N′)y and positions 2-3, 4-5, 6-7, 13-14, 15-16 and 17-18 of (N)x;
f) an internucleotide modification between nucleotides at positions 1-2, 3-4, 16-17 and 18-19 of the (N′)y and 2-3, 4-5, 15-16 and 17-18 of the antisense strand;
g) an internucleotide modification between nucleotides at positions 1-2, 3-4, 16-17 and 18-19 of (N′)y and 2-3 and 17-18 of (N)x;
h) an internucleotide modification between nucleotides at positions 2-3 and 17-18 of (N′)y and 1-2, 3-4, 16-17 and 18-19 of (N)x;
i) an internucleotide modification between nucleotides at positions 1-2, 6-7, 13-14 and 18-19 of (N′)y and 5-6 and 14-15 of (N)x;
j) an internucleotide modification between nucleotides at positions 6-7 and 13-14 of (N′)y and 1-2, 5-6, 14-15 and 18-19 of (N)x;
k) an internucleotide modification between nucleotides at positions 1-2, 4-5, 6-7, 13-14, 15-16 and 18-19 of (N′)y and 5-6, 7-8, 12-13 and 14-15 of (N)x; and
l) an internucleotide modification between nucleotides at positions 4-5, 6-7, 13-14 and 15-16 of the (N′)y and 1-2, 5-6, 7-8, 12-13, 14-15 and 18-19 of (N)x.

Further, the alpha phosphate modification may be selected from the group consisting of phospho-ethyl triester, phospho-propyl triester and phospho-butyl trimester.

Additionally, the internucleotide linkage modification may be a 5′2′ bridge.

Further, one of (N)x and (N′)y may comprise one or more L-nucleotide, optionally having any one of the following motifs:
a) an L-nucleotide at each of positions 1-19 of the (N′)y;
b) an L-nucleotide at each of positions 1-19 of the (N)x;
c) an L-nucleotide at each of positions 9-11 of the (N′)y and at each of positions 1-9 and 11-19 of (N)x;
d) an L-nucleotide at each of positions 1-8, and 12-19 of the (N′)y and each of positions 2-9 and 11-18 of (N)x;
e) an L-nucleotide at each of positions 1-8 and 14-19 of (N′)y and each of positions 2-7 and 13-18 of (N)x;
f) an L-nucleotide at each of positions 1-4 and 16-19 of the (N′)y and each of positions 2-5 and 15-18 of (N)x;
g) an L-nucleotide at each of positions 1-4, 16-19 of (N′)y and each of positions 2-3 and 17-18 of (N)x;
h) an L-nucleotide at each of positions 2-3 and 17-18 of (N′)y and each of positions 1-4 and 16-19 of (N)x;
i) an L-nucleotide at each of positions 1-2, 6-7, 13-14 and 18-19 of (N′)y and each of positions 5-6 and 14-15 of (N)x;
j) an L-nucleotide at each of positions 6-7 and 13-14 of (N′)y and each of positions 1-2, 5-6, 14-15 and 18-19 of (N)x;
k) an L-nucleotide at each of positions 1-2, 4-7, 13-16 and 18-19 of (N′)y and each of 5-8, 12-15 of (N)x;
l) an L-nucleotide at each of positions 4-7 and 13-16 of (N′)y and each of positions 1-2, 5-8, 12-15 and 18-19 of (N)x.

In certain embodiments concerning all the above compounds, x=y, optionally x=y=19 or x=y=20 or x=y=21 or x=y=22 or x=y=23.

In certain embodiments Z═Z′=0.

In additional embodiments, the compound may further comprise one or more modified nucleotides. The modified nucleotide may be selected from the group consisting of DNA, LNA, PNA or arabinoside.

In additional embodiments, the compound may further comprise one or more sugar modifications. The sugar modification may be selected from the group consisting of 2′ fluoro, 2′Oallyl, 2′ amine and 2′ alkoxy. Further, the 2′ alkoxy may be 2′O-methyl.

In additional embodiments, the compound may further comprise one or more terminal modifications. The terminal modification may be selected from the group consisting of a nucleotide, a di-nucleotide, an oligonucleotide a lipid, a peptide, a sugar, and an amine.

The present invention further provides for an siRNA produced by cleavage of any one of the inhibitory compounds as disclosed herein. The cleavage may optionally be nuclease cleavage.

In addition, the present invention further provides for a pharmaceutical composition comprising any one of the inhibitory compound disclosed herein, and a method of treating any one of the diseases and conditions disclosed herein by administering to a patient in need thereof a therapeutically effective amount of any one of said pharmaceutical compositions.

Another embodiment which is envisaged is a longer molecule comprised of a longer sequence which encodes a molecule comprising an siRNA which is produced via internal cellular processing of the longer molecule, e.g., long dsRNAs.

Additionally, specifications of the siRNA molecules used in the present invention may provide an oligoribonucleotide wherein the dinucleotide dTdT is covalently attached to the 3′ terminus, and/or in at least one nucleotide a sugar residue is modified, possibly with a modification comprising a 2′-O-Methyl modification. Further, the 2′ OH group may be replaced by a group or moiety selected from the group comprising —OCH₃, —OCH₂CH₃, —OCH₂CH₂CH₃, —O—CH₂CHCH₂, —NH₂, and F. Further, the preferable compounds of the present invention as disclosed above may be phosphorylated or non-phosphorylated.

Additionally, the siRNA used in the present invention may be an oligoribonucleotide wherein in alternating nucleotides modified sugars are located in both strands. Particularly, the oligoribonucleotide may comprise one of the sense strands wherein the sugar is unmodified in the terminal 5′ and 3′ nucleotides, or one of the antisense strands wherein the sugar is modified in the terminal 5′ and 3′ nucleotides.

As detailed above, possible modification of the molecules of the present invention include modification of a sugar moiety, optionally at the 2′ position, whereby the 2′ OH group is replaced by a group or moiety selected from the group comprising —H—OCH₃, —OCH₂CH₃, —OCH₂CH₂CH₃, —O—CH₂CHCH₂, —NH₂, and —F.

Further possible modifications include modification of the nucleobase moiety and the modification or modified nucleobase may be selected from the group comprising inosine, xanthine, hypoxanthine, 2-aminoadenine, 6-methyl, 2-propyl and other alkyladenines, 5-halo-uracil, 5-halo-cytosine, 5-halo-cytosine, 6-aza-cytosine, 6-aza-thymine, pseudouracil, 4-thio-uracil, 8-halo-adenine, 8-amino-adenine, 8-thiol-adenine, 8-thioalkyl-adenines, 8-hydroxyl-adenine and other 8-substituted adenines, 8-halo-guanines, 8-amino-guanine, 8-thiol-guanine, 8-thioalkyl-guanine, 8-hydroxyl-guanine and other substituted guanines, other aza- and deaza adenines, other aza- and deaza guanines, 5-trifluoromethyl-uracil and 5-trifluoro-cytosine.

In an additional embodiment, the modification is a modification of the phosphate moiety, whereby the modified phosphate moiety is selected from the group comprising phosphothioate or lack of a phosphate group.

The molecules of the present invention may comprise siRNAs, synthetic siRNAs, shRNAs and synthetic shRNAs, in addition to other nucleic acid sequences or molecules which encode such molecules or other inhibitory nucleotide molecules. As used herein, in the description of any strategy for the design of molecules, RNAi or any embodiment of RNAi disclosed herein, the term “end modification” means a chemical entity added to the most 5′ or 3′ nucleotide of the first and/or second strand. Examples for such end modifications include, but are not limited to, 3′ or 5′ phosphate, inverted abasic, abasic, amino, fluoro, chloro, bromo, CN, CF₃, methoxy, imidazolyl, carboxylate, phosphothioate, C₁ to C₂₂ and lower alkyl, lipids, sugars and polyaminoacids (i.e. peptides), substituted lower alkyl, alkaryl or aralkyl, OCF₃, OCN, O—, S—, or N-alkyl; O—, S—, or N-alkenyl; SOCH₃; SO₂CH₃; ONO₂; NO₂, N₃; heterocycloalkyl; heterocycloalkaryl; aminoalkylamino; polyalkylamino or substituted silyl, as, among others, described in European patents EP 0 586 520 B1 or EP 0 618 925 B1.

A further end modification is a biotin group. Such biotin group may preferably be attached to either the most 5′ or the most 3′ nucleotide of the first and/or second strand or to both ends. In a more preferred embodiment the biotin group is coupled to a polypeptide or a protein. It is also within the scope of the present invention that the polypeptide or protein is attached through any of the other aforementioned end modifications.

The various end modifications as disclosed herein are preferably located at the ribose moiety of a nucleotide of the nucleic acid according to the present invention. More particularly, the end modification may be attached to or replace any of the OH-groups of the ribose moiety, including but not limited to the 2′OH, 3′OH and 5′OH position, provided that the nucleotide thus modified is a terminal nucleotide. Inverted abasic or abasic are nucleotides, either desoxyribonucleotides or ribonucleotides which do not have a nucleobase moiety. This kind of compound is, among others, described in Sternberger, et al.,. (2002). Antisense Nucleic Acid Drug Dev, 12, 131-43.

Further modifications can be related to the nucleobase moiety, the sugar moiety or the phosphate moiety of the individual nucleotide.

Such modification of the nucleobase moiety can be such that the derivatives of adenine, guanine, cytosine and thymidine and uracil, respectively, are modified. Particularly preferred modified nucleobases are selected from the group comprising inosine, xanthine, hypoxanthine, 2-aminoadenine, 6-methyl, 2-propyl and other alkyladenines, 5-halo-uracil, 5-halo-cytosine, 5-halo-cytosine, 6-aza-cytosine, 6-aza-thymine, pseudouracil, 4-thio-uracil, 8-halo-adenine, 8-amino-adenine, 8-thiol-adenine, 8-thioalkyl-adenines, 8-hydroxyl-adenine and other 8-substituted adenines, 8-halo-guanines, 8-amino-guanine, 8-thiol-guanine, 8-thioalkyl-guanine, 8-hydroxyl-guanine and other substituted guanines, other aza- and deaza adenines, other aza- and deaza guanines, 5-trifluoromethyl-uracil and 5-trifluoro-cytosine. In another preferred embodiment, the sugar moiety of the nucleotide is modified, whereby such modification preferably is at the 2′ position of the ribose and desoxyribose moiety, respectively, of the nucleotide. More preferably, the 2′ OH group is replaced by a group or moiety selected from the group comprising amino, fluoro, alkoxy and alkyl. alkoxy may be either methoxy or ethoxy; alkyl may be methyl, ethyl, propyl, isobutyl, butyl or isobutyl.

A further form of nucleotides used may actually be siNA which is, among others, described in international patent application WO 03/070918.

It is to be understood that, in the context of the present invention, any of the siRNA molecules disclosed herein, or any long double-stranded RNA molecules (typically 25-500 nucleotides in length) which are processed by endogenous cellular complexes (such as DICER—see above) to form the siRNA molecules disclosed herein, or molecules which comprise the siRNA molecules disclosed herein, can be incorporated into the molecules of the present invention to form additional novel molecules, and can employed in the treatment of the diseases or disorders described herein.

In particular, it is envisaged that a long oligonucleotide (typically about 80-500 nucleotides in length) comprising one or more stem and loop structures, where stem regions comprise the oligonucleotides of the invention, may be delivered in a carrier, preferably a pharmaceutically acceptable carrier, and may be processed intracellularly by endogenous cellular complexes (e.g. by DROSHA and DICER as described above) to produce one or more smaller double stranded oligonucleotides (siRNAs) which are oligonucleotides of the invention. This oligonucleotide can be termed a tandem shRNA construct. It is envisaged that this long oligonucleotide is a single stranded oligonucleotide comprising one or more stem and loop structures, wherein each stem region comprises a sense and corresponding antisense siRNA sequence. Any molecules, such as, for example, antisense DNA molecules which comprise the inhibitory sequences disclosed herein (with the appropriate nucleic acid modifications) are particularly desirable and may be used in the same capacity as their corresponding RNAs/siRNAs for all uses and methods disclosed herein.

By the term “antisense” (AS) or “antisense fragment” is meant a polynucleotide fragment (comprising either deoxyribonucleotides, ribonucleotides, synthetic or modified nucleotides or a mixture thereof) having inhibitory antisense activity, said activity causing a decrease in the expression of the endogenous genomic copy of the corresponding gene. The sequence of the AS is designed to complement a target mRNA of interest and form an RNA:AS duplex. This duplex formation can prevent processing, splicing, transport or translation of the relevant mRNA. Moreover, certain AS nucleotide sequences can elicit cellular RNase H activity when hybridized with the target mRNA, resulting in mRNA degradation (Calabretta et al, 1996: Semin Oncol. 23(1):78-87). In that case, RNase H will cleave the RNA component of the duplex and can potentially release the AS to further hybridize with additional molecules of the target RNA. An additional mode of action results from the interaction of AS with genomic DNA to form a triple helix which can be transcriptionally inactive.

All analogues of, or modifications to, a nucleotide/oligonucleotide may be employed with the present invention, provided that said analogue or modification does not substantially affect the function of the nucleotide/oligonucleotide. The nucleotides can be selected from naturally occurring or synthetic modified bases. Naturally occurring bases include adenine, guanine, cytosine, thymine and uracil. Modified bases of nucleotides include inosine, xanthine, hypoxanthine, 2-aminoadenine, 6-methyl, 2-propyl and other alkyl adenines, 5-halo uracil, 5-halo cytosine, 6-aza cytosine and 6-aza thymine, psuedo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

In addition, analogues of polynucleotides can be prepared wherein the structure of the nucleotide is fundamentally altered and that are better suited as therapeutic or experimental reagents. An example of a nucleotide analogue is a peptide nucleic acid (PNA) wherein the deoxyribose (or ribose) phosphate backbone in DNA (or RNA is replaced with a polyamide backbone which is similar to that found in peptides. PNA analogues have been shown to be resistant to degradation by enzymes and to have extended lives in vivo and in vitro. Further, PNAs have been shown to bind stronger to a complementary DNA sequence than a DNA molecule. This observation is attributed to the lack of charge repulsion between the PNA strand and the DNA strand. Other modifications that can be made to oligonucleotides include polymer backbones, cyclic backbones, or acyclic backbones.

By “homolog/homology”, as utilized in the present invention, is meant at least about 70%, preferably at least about 75% homology, advantageously at least about 80% homology, more advantageously at least about 90% homology, even more advantageously at least about 95%, e.g., at least about 97%, about 98%, about 99% or even about 100% homology. The invention also comprehends that these nucleotides/oligonucleotides/polynucleotides can be used in the same fashion as the herein or aforementioned polynucleotides and polypeptides.

Alternatively or additionally, “homology”, with respect to sequences, can refer to the number of positions with identical nucleotides, divided by the number of nucleotides in the shorter of the two sequences, wherein alignment of the two sequences can be determined in accordance with the Wilbur and Lipman algorithm ((1983) Proc. Natl. Acad. Sci. USA 80:726); for instance, using a window size of 20 nucleotides, a word length of 4 nucleotides, and a gap penalty of 4, computer-assisted analysis and interpretation of the sequence data, including alignment, can be conveniently performed using commercially available programs (e.g., Intelligenetics™ Suite, Intelligenetics Inc., CA). When RNA sequences are said to be similar, or to have a degree of sequence identity or homology with DNA sequences, thymidine (T) in the DNA sequence is considered equal to uracil (U) in the RNA sequence. RNA sequences within the scope of the invention can be derived from DNA sequences or their complements, by substituting thymidine (T) in the DNA sequence with uracil (U).

Additionally or alternatively, amino acid sequence similarity or homology can be determined, for instance, using the BlastP program (Altschul et al., Nucl. Acids Res. 25:3389-3402) and available at NCBI. The following references provide algorithms for comparing the relative identity or homology of amino acid residues of two polypeptides, and additionally, or alternatively, with respect to the foregoing, the teachings in these references can be used for determining percent homology: Smith et al., (1981) Adv. Appl. Math. 2:482-489; Smith et al., (1983) Nucl. Acids Res. 11:2205-2220; Devereux et al., (1984) Nucl. Acids Res. 12:387-395; Feng et al., (1987) J. Molec. Evol. 25:351-360; Higgins et al., (1989) CABIOS 5:151-153; and Thompson et al., (1994) Nucl. Acids Res. 22:4673-4680.

“Having at least X % homology”—with respect to two amino acid or nucleotide sequences, refers to the percentage of residues that are identical in the two sequences when the sequences are optimally aligned. Thus, 90% amino acid sequence identity means that 90% of the amino acids in two or more optimally aligned polypeptide sequences are identical.

The invention has been described in an illustrative manner, and it is to be understood that the terminology used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

Throughout this application, various publications, including United States patents, are referenced by author and year and patents by number. The disclosures of these publications and patents and patent applications in their entireties are hereby incorporated by reference into this application in order to more fully describe the state of the art to which this invention pertains.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988).

Standard organic synthesis protocols known in the art not specifically described herein are generally followed essentially as in Organic syntheses: Vol-79, editors vary, J. Wiley, New York, (1941-2003); Gewert et al., Organic synthesis workbook, Wiley-VCH, Weinheim (2000); Smith & March, Advanced Organic Chemistry, Wiley-Interscience; 5th edition (2001).

Standard medicinal chemistry methods known in the art not specifically described herein are generally followed essentially as in the series “Comprehensive Medicinal Chemistry”, by various authors and editors, published by Pergamon Press.

The features of the present invention disclosed in the specification, the claims and/or the drawings may both separately and in any combination thereof be material for realizing the invention in various forms thereof.

### Example 1

### General Materials and Methods

If not indicated to the contrary, the following materials and methods were used in

### Examples 1-5

### Cell Culture

The first human cell line, namely HeLa cells (American Type Culture Collection) were cultured as follows: Hela cells (American Type Culture Collection) were cultured as described in Czauderna F et al. (Czaudema, F., Fechtner, M., Aygun, H., Arnold, W., Klippel, A., Giese, K. & Kaufmann, J. (2003). Nucleic Acids Res, 31, 670-82).

The second human cell line was a human keratinozyte cell line which was cultivated as follows: Human keratinocytes were cultured at 37° C. in Dulbecco's modified Eagle medium (DMEM) containing 10% FCS.

The mouse cell line was B16V (American Type Culture Collection) cultured at 37° C. in Dulbecco's modified Eagle medium (DMEM) containing 10% FCS. Culture conditions were as described in Methods Find Exp Clin Pharmacol. 1997 May; 19(4):231-9:

In each case, the cells were subject to the experiments as described herein at a density of about 50,000 cells per well and the double-stranded nucleic acid according to the present invention was added at 20 nM, whereby the double-stranded nucleic acid was complexed using 1 μg/ml of a proprietary lipid as described below.

### Induction of Hypoxia-Like Conditions

The cells were treated with CoCl₂ for inducing a hypoxia-like condition as follows: siRNA transfections were carried out in 10-cm plates (30-50% confluency) as described by Czauderna et al., 2003; Kretschmer et al., 2003. Briefly, siRNA were transfected by adding a preformed 10× concentrated complex of GB and lipid in serum-free medium to cells in complete medium. The total transfection volume was 10 ml. The final lipid concentration was 1.0 μg/ml; the final siRNA concentration was 20 nM unless otherwise stated. Induction of the hypoxic responses was carried out by adding CoCl₂ (100 μM) directly to the tissue culture medium 24 h before lysis.

### Preparation of Cell Extracts and Immuno Blotting

The preparation of cell extracts and immuno blot analysis are carried out essentially as described by Klippel et al. (Klippel, et al., (1998). Mol Cell Biol, 18, 5699-711; Klippel, et al., (1996). Mol Cell Biol, 16, 4117-27).

### Example 2

### Preparation of Nucleic Acid Molecules/siRNAs

The molecules and compounds of the present invention can be synthesized by any of the methods which are well-known in the art for synthesis of ribonucleic (or deoxyribonucleic) oligonucleotides. For example, a commercially available machine (available, inter alia, from Applied Biosystems) can be used; the oligonucleotides are prepared according to the sequences disclosed herein.

The strands are synthesized separately and then are annealed to each other in the tube.

The molecules of the invention may be synthesized by procedures known in the art e.g. the procedures as described in Usman et al., 1987, J. Chem. Soc., 109, 7845; Scaringe et al., 1990, Nucleic Acids Res., 18, 5433; Wincott et al., 1995, Nucleic Acids Res. 23, 2677-2684; and Wincott et al., 1997, Methods Mol. Bio., 74, 59, and may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired.

The linker can be a polynucleotide linker or a non-nucleotide linker.

For further information, see PCT publication No. WO 2004/015107 (atugen).

### Example 3

### Pharmacology and Drug Delivery

The nucleotide sequences of the present invention can be delivered directly and must be rendered nuclease resistant e.g. by modification as disclosed herein. The compounds or pharmaceutical compositions of the present invention are administered and dosed in accordance with good medical practice, taking into account the clinical condition of the individual patient, the disease to be treated, the site and method of administration, scheduling of administration, patient age, sex, body weight and other factors known to medical practitioners.

The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

The treatment generally has a length proportional to the length of the disease process and drug effectiveness and the patient species being treated. It is noted that humans are treated generally longer than the mice or other experimental animals exemplified herein.

The compounds of the present invention can be administered by any of the conventional routes of administration. It should be noted that the compound can be administered as the compound or as pharmaceutically acceptable salt and can be administered alone or as an active ingredient in combination with pharmaceutically acceptable carriers, solvents, diluents, excipients, adjuvants and vehicles. The compounds can be administered topically, orally, subcutaneously or parenterally including intravenous, intraarterial, intramuscular, intraperitoneally, and intranasal administration as well as intrathecal and infusion techniques. Implants of the compounds are also useful. Liquid forms may be prepared for injection, the term including subcutaneous, transdermal, intravenous, intramuscular, intrathecal, and other parental routes of administration. The liquid compositions include aqueous solutions, with and without organic cosolvents, aqueous or oil suspensions, emulsions with edible oils, as well as similar pharmaceutical vehicles. In addition, under certain circumstances the compositions for use in the novel treatments of the present invention may be formed as aerosols, for intranasal and like administration. The patient being treated is a warm-blooded animal and, in particular, mammals including man. The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the invention.

When administering the compound of the present invention parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and an oil, especially a vegetable oil and a lipid and suitable mixtures thereof.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used has to be compatible with the compounds.

Sterile injectable solutions can be prepared by incorporating the compounds utilized in practicing the present invention in the required amount of the appropriate solvent with several of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, iontophoretic, polymer matrices, liposomes, and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

In a particular embodiment, the administration comprises intravenous (i.v.) administration. In another embodiment the administration comprises topical or local administration, particularly to the eye or nasal passage for a local or systemic effect. In addition, in certain embodiments the compositions for use in treating disorders of the eye or CNS may be formulated for intranasal administration.

In some embodiments the compounds are useful in the treatment of an eye disorder. The compounds can be administered directly to the eye, for example in the form of eye drops, gel or ointment, for local or systemic delivery to the target cell. Accordingly, the compounds are incorporated into topical ophthalmic formulations. The compounds may be combined with opthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving a compound in a physiologically acceptable isotonic aqueous buffer. Preferred additives for use in sterile, isotonic solutions include, but are not limited to, benzalkonium chloride, thimerosal, chlorobutanol, sodium chloride, boric acid and mixtures thereof. In some embodiments, benzalkonium chloride or thimerosal is added as an antimicrobial preservative.

Ophthalmic formulations may contain an agent to increase solubility, (such as a surfactant), or viscosity, (i.e., hydroxymethylcellulose, hydroxyethylcellulose, hydroxypropylmethylcellulose, methylcellulose, polyvinylpyrrolidone, or the like,) to improve the retention of the formulation in the conjunctival sac. Gelling agents can also be used, including, but not limited to, gellan and xanthan gum. In various embodiments an ophthalmic ointment is preferred. A sterile ophthalmic ointment formulation may be prepared by combining the active ingredient with an appropriate vehicle, such as, mineral oil, liquid lanolin, or white petrolatum and optionally a preservative. Topical formulations can include from about 0.001% by weight to about 10% by weight of the active compound with the remainder of the formulation being the carrier and other materials known in the art as topical pharmaceutical components.

Eye drops comprising a compound, in particular an siRNA compound of the present invention, are useful in targeting genes expressed in various cells and tissues of the eye. Accordingly, eye drops comprising a compound of the present invention are useful in treating age related macular degeneration, diabetic retinopathy, glaucoma.

In another embodiment, the compounds can be administered directly to the nasal passage, for example in the form of nose drops, nasal spray, aerosol, gel or ointment, for local or systemic delivery to the target cell. Intranasal administration of a formulation comprising a compound of the present invention is useful in treating age related macular degeneration, diabetic retinopathy.

In some embodiments the compounds of the present invention are useful for treating disorders in the inner ear. Administration of the compound to the inner ear is effected by intratympanic delivery, systemic administration or alternatively via intranasal administration. A pharmacological formulation of the compound utilized in the present invention can be administered orally to the patient. Conventional methods such as administering the compound in tablets, suspensions, solutions, emulsions, capsules, powders, syrups and the like are usable. Known techniques which deliver it orally or intravenously and retain the biological activity are preferred. In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used.

In general, the active dose of compound for humans is in the range of from 1 ng/kg to about 20-100 mg/kg body weight per day, preferably about 0.01 mg to about 2-10 mg/kg body weight per day, in a regimen of one dose per day or twice or three or more times per day for a period of 1-2 weeks or longer, preferably for 24 to 48 hrs or by continuous infusion during a period of 1-2 weeks or longer.

### Administration of Compounds of the Present Invention to the Eye

The compounds of the present invention can be administered to the eye topically eg in the form of eye drops, gel or an ointment or by an injection, such as an intravitreal injection, a sub-retinal injection or a bilateral injection.

Further information on administration of the compounds of the present invention can be found in Tolentino et al., Retina 24 (2004) 132-138; Reich et al., Molecular vision 9 (2003) 210-216.

### Pulmonary Administration of Compounds of the Present Invention

The therapeutic compositions of the present invention are preferably administered into the lung by inhalation of an aerosol containing such composition/compound, or by intranasal or intratracheal instillation of said compositions. Formulating the compositions in liposomes may benefit absorption. Additionally, the compositions may include a PFC liquid such as perflubron, and the compositions may be formulated as a complex of the compounds of the invention with polyethylemeimine (PEI).

For further information on pulmonary delivery of pharmaceutical compositions see Weiss et al., Human gene therapy 10:2287-2293 (1999); Densmore et al., Molecular therapy 1:180-188 (1999); Gautam et al., Molecular therapy 3:551-556 (2001); and Shahiwala & Misra, AAPS PharmSciTech 5 (2004). Additionally, respiratory formulations for siRNA are described in U.S. patent application No. 2004/0063654 of Davis et el.

### Administration of Compounds of the Present Invention to the Ear

A preferred administration mode is directly to the affected portion of the ear or vestibule, topically as by implant for example, and, preferably to the affected hair cells or their supporting cells, so as to direct the active molecules to the source and minimize its side effects. A preferred administration mode is a topical delivery of the inhibitor(s) onto the round window membrane of the cochlea. Such a method of administration of other compounds is disclosed for example in Tanaka et al. (Hear Res. 2003 March; 177(1-2):21-31).

Additional modes of administration to the ear are by administration of liquid drops to the ear canal, delivery to the scala tympani chamber of the inner ear by transtympanic injection, or provision as a diffusible member of a cochlear hearing implant.

In the treatment of pressure sores or other wounds, the administration of the pharmaceutical composition is preferably by topical application to the damaged area, but the compositions may also be administered systemically.

Additional formulations for improved delivery of the compounds of the present invention can include non-formulated compounds, compounds covalently bound to cholesterol, and compounds bound to targeting antibodies (Song et al., Nat. Biotechnol. 2005 June; 23(6):709-17).

1. A compound having a structure set forth below:

| | |
|---|---|
| 5′ (N)ₓ-Z 3′ | (antisense strand) |
| 3′ Z′-(N′)_{y} 5′ | (sense strand) |

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein each of x and y is an integer between 19 and 23;
wherein each of Z and Z′ may be present or absent, but if present is 1-5 consecutive nucleotides covalently attached at the 3′ terminus of the strand in which it is present;
wherein the sequence of (N)x is complementary to the sequence of (N′)y;
wherein the sequence of (N′)y is present within the mRNA of a target gene;
wherein at least one of (N)x or (N′)y comprises a modification selected from the group consisting of internucleotide modification and an L-nucleotide;
and wherein the internucleotide modification is 5′-2′ bridge or an alpha phosphate modification selected from the group consisting of thiophosphate and triester.

2. The compound according to claim 1 wherein x=y=19 and the alpha phosphate modification is present in any one of the motifs selected from the group consisting of
a) an internucleotide modification between nucleotides at positions 1-19 of (N′)y;
b) an internucleotide modification between nucleotides at positions 1-19 of (N)x;
c) an internucleotide modification between nucleotides at positions 9-11 of (N′)y and positions 1-9 and 11-19 of (N)x;
d) an internucleotide modification between nucleotides at positions 1-2, 3-4, 5-6, 7-8, 12-13, 14-15, 16-17 and 18-19 of (N′)y and positions 2-3, 4-5, 6-7, 8-9 11-12, 13-14, 15-16 and 17-18 of (N)x;
e) an internucleotide modification between nucleotides at positions 1-2, 3-4, 5-6, 7-8, 14-15, 16-17 and 18-19 of (N′)y and positions 2-3, 4-5, 6-7, 13-14, 15-16 and 17-18 of (N)x;
f) an internucleotide modification between nucleotides at positions 1-2, 3-4, 16-17 and 18-19 of the (N′)y and 2-3, 4-5, 15-16 and 17-18 of the antisense strand;
g) an internucleotide modification between nucleotides at positions 1-2, 3-4, 16-17 and 18-19 of (N′)y and 2-3 and 17-18 of (N)x;
h) an internucleotide modification between nucleotides at positions 2-3 and 17-18 of (N′)y and 1-2, 3-4, 16-17 and 18-19 of (N)x;
i) an internucleotide modification between nucleotides at positions 1-2, 6-7, 13-14 and 18-19 of (N′)y and 5-6 and 14-15 of (N)x;
j) an internucleotide modification between nucleotides at positions 6-7 and 13-14 of (N′)y and 1-2, 5-6, 14-15 and 18-19 of (N)x;
k) an internucleotide modification between nucleotides at positions 1-2, 4-5, 6-7, 13-14, 15-16 and 18-19 of (N′)y and 5-6, 7-8, 12-13 and 14-15 of (N)x; and
l) an internucleotide modification between nucleotides at positions 4-5, 6-7, 13-14 and 15-16 of the (N′)y and 1-2, 5-6, 7-8, 12-13, 14-15 and 18-19 of (N)x.

3. The compound according to claim 1 wherein the alpha phosphate modification is a thiophosphate.

4. The compound according to claim 1 wherein the internucleotide linkage modification is a 5′-2′ bridge.

5. The compound according to claim 1 wherein one of (N)x and (N′)y comprise one or more L-nucleotide.

6. The compound according to claim 1 wherein x=y=19 and which has any one of the following motifs selected from the group consisting of:
a) an L-nucleotide at each of positions 1-19 of the (N′)y;
b) an L-nucleotide at each of positions 1-19 of the (N)x;
c) an L-nucleotide at each of positions 9-11 of the (N′)y and at each of positions 1-9 and 11-19 of (N)x;
d) an L-nucleotide at each of positions 1-8, and 12-19 of the (N′)y and each of positions 2-9 and 11-18 of (N)x;
e) an L-nucleotide at each of positions 1-8 and 14-19 of (N′)y and each of positions 2-7 and 13-18 of (N)x;
f) an L-nucleotide at each of positions 1-4 and 16-19 of the (N′)y and each of positions 2-5 and 15-18 of (N)x;
g) an L-nucleotide at each of positions 1-4, 16-19 of (N′)y and each of positions 2-3 and 17-18 of (N)x;
h) an L-nucleotide at each of positions 2-3 and 17-18 of (N′)y and each of positions 1-4 and 16-19 of (N)x;
i) an L-nucleotide at each of positions 1-2, 6-7, 13-14 and 18-19 of (N′)y and each of positions 5-6 and 14-15 of (N)x;
j) an L-nucleotide at each of positions 6-7 and 13-14 of (N′)y and each of positions 1-2, 5-6, 14-15 and 18-19 of (N)x;
k) an L-nucleotide at each of positions 1-2, 4-7, 13-16 and 18-19 of (N′)y and each of 5-8, 12-15 of (N)x; and
l) an L-nucleotide at each of positions 4-7 and 13-16 of (N′)y and each of positions 1-2, 5-8, 12-15 and 18-19 of (N)x.

7. The compound of claim 1 wherein x=y.

8. The compound of claim 7 wherein x=y=19 or x=y=23.

9. The compound of claim 1 wherein each of Z and Z′ is absent.

10. The compound of claim 1 wherein at least one of (N)x and (N′)y further comprises one or more modified nucleotides.

11. The compound of claim 10 wherein the modified nucleotide is selected from the group consisting of DNA, LNA, PNA and arabinoside.

12. The compound of claim 10 wherein the modified nucleotide comprises a sugar modifications.

13. The compound of claim 12 wherein the sugar modification is selected from the group consisting of 2′ fluoro, 2′O allyl, 2′ amine and 2′ alkoxy.

14. The compound according to claim 13 wherein the 2′ alkoxy is 2′O-methyl.

15. The compound of claim 1 further comprising one or more terminal modifications.

16. The compound of claim 15 wherein the terminal modification is selected from the group consisting of a nucleotide, a di-nucleotide, an oligonucleotide a lipid, a peptide, a sugar, and an amine.

17. A siRNA produced by cleavage of the compound of claim 1.

18. The siRNA of claim 17 wherein the cleavage is nuclease cleavage.

19. A pharmaceutical composition comprising at least one compound of claims 1.
